(19) United States
(12) Patent Application Publication
(10) Pub. No.: US 2016/0106954 A1
(43) Pub. Date: Apr. 21, 2016

(54) CATHETER SYSTEM

(71) Applicant: P & M HEBBARD PTY LTD, Wangaratta, Victoria (AU)

(72) Inventor: Peter HEBBARD, Wangaratta (AU)

(21) Appl. No.: 14/892,041

(22) PCT Filed: Mar. 12, 2014
(86) PCT No.: PCT/AU2014/000248

§ 371 Date: Nov. 18, 2015

(30) Foreign Application Priority Data
May 27, 2013 (AU) 2013901874
Nov. 28, 2013 (AU) 2013101567

(51) Int. Cl. A61M 25/01

(52) U.S. Cl. CPC A61M 25/0102

(57) ABSTRACT

A catheter system including a catheter (10) and a stylet (30). The stylet includes an elongate portion (31) and a shaped portion (32). The elongate portion is receivable within the catheter. The elongate portion and the catheter are co-operably configured such that, when the elongate portion is so received, at least a portion of the elongate portion is spaced from the catheter such that the at least portion and the catheter together define at least one elongate void for conveying fluid along the catheter at least one of to or from one or more sites within a patient. The shaped portion is positioned to be within the patient.

### FIELD

Various aspects of the invention relate to catheter systems and related methods and components therefor.

### BACKGROUND

A catheter is a tube insertable into a patient, so that part of the tube is in the patient and another part is external the patient, to establish fluid communication with one or more sites within the patient. By way of example, nerve block catheters are used to deliver anaesthetic to nerves whereas other catheters are used for withdrawing blood samples, administering medication into the blood stream or measuring pressure.

To so measure blood pressure, the catheter is inserted into a blood vessel and connected to a blood pressure transducer (or blood pressure “sensor”—the terms are used interchangeably herein) to establish fluid communication between the vessel and the transducer. A typical blood pressure transducer contains a membrane which moves with pressure changes. This movement then generates an electrical signal which is translated into the measured pressure displayed on the monitor on the transducer. During arterial pressure measurement, the compliance of commercial transducers is such that blood moves in and out of the end of the catheter during the beat-to-beat pressure change of the pulse.

Some existing catheters take the form of a simple tube being open at both of its ends, whereas others have a closed distal end and a set of side openings.

Nerve block catheters are conventionally inserted by:
1. inserting into the patient a tubular needle;
2. threading the catheter through the tubular needle;
3. withdrawing the tubular needle leaving a free open end of the catheter projecting from the patient; and
4. fitting a connector to the projecting free end of the catheter to connect the catheter to a fluid source e.g. a syringe loaded with anaesthetic (whereas other catheters might be connected to a fluid destination e.g. a vacuum source for drawing a blood sample).

The present inventor has recognised that the proper functioning of catheters is sometimes adversely impacted by the catheter kinking, the buildup of material (e.g. blood clotting), leakage of injected fluid back along the exterior of the catheter and/or by the catheter inadvertently (partly or wholly) being withdrawn from the patient. Accordingly the various aspects of the present invention aim to at least partly address one or more of these problems, or at least to provide alternatives for those concerned with catheter systems and their use.

It is not admitted that any of the information in this patent specification is common general knowledge, or that the person skilled in the art could be reasonably expected to ascertain or understand it, regard it as relevant or combine it in any way at the priority date.

### SUMMARY

One aspect of the invention provides a method, of fluidly communicating with a blood vessel within a patient, including

inserting into the blood vessel a catheter; and

inserting into the catheter a stylet configured such that, when so inserted, at least a portion of the stylet is spaced from the catheter such that the at least portion and the catheter together define at least one elongate void for conveying fluid along the catheter at least one of to or from the blood vessel.

The blood vessel may be an artery.

Another aspect of the invention provides a method of monitoring blood pressure including so fluidly communicating with the blood vessel. This method preferably includes delivering fluid, e.g. saline, via the catheter to the blood vessel to resist clotting.

The stylet may include a shaped portion positioned to be within the patient and shaped to act upon to remove build-up from the catheter. Preferably the shaped portion has a shape complementary to a cylindrical interior of the catheter, and most preferably is shaped to obstruct flow through the catheter.

The method may further include manipulating the stylet to remove build-up, from the catheter, within the patient, e.g. to remove build-up, from an open end of the catheter.

Preferably the method includes, after inserting the stylet, leaving for a period of time the stylet in place. Preferably the period is at least one hour. Optionally the inserting into the patient a catheter and the inserting into the catheter a stylet are during an attendance to the patient; and the method further includes leaving the stylet and catheter in place until a subsequent attendance to the patient.

The stylet may be replaced with another stylet.

The inserting into the patient a catheter preferably includes inserting into the patient a needle externally carrying the catheter; and withdrawing the needle.

Another aspect of the invention provides a catheter system including

a catheter; and

a stylet;

the stylet including an elongate portion and a shaped portion;

the elongate portion being receivable within the catheter;

the elongate portion and the catheter being co-operably configured such that, when the elongate portion is so received, at least a portion of the elongate portion is spaced from the catheter such that the at least portion and the catheter together define at least one elongate void for conveying fluid along the catheter at least one of to or from one or more sites within a patient;

the shaped portion being positioned to be within the patient.

Preferably the shaped portion is shaped to obstruct the catheter. The shaped portion may be a bulbous portion and is preferably in substance at an end of the stylet.

The catheter may have an open end positionable within the patient in which case the stylet is preferably advanceable within the catheter to move the shaped portion at least to the open end, when the open end is within the patient, to remove build-up at the open end.

It is preferred that at least a rearward portion of an exterior of the shaped portion rearwardly converges.

Another aspect of the invention provides a catheter system including

a catheter; and

a stylet;

the stylet including an elongate portion;

the elongate portion being receivable within the catheter;

the catheter having an open end positionable within the patient; and

the stylet being long enough to be manipulated external the patient to remove build-up from the open end within the patient.

The system may further include a needle, e.g. a short beveled nerve block needle, for inserting the catheter into the patient, in which case the needle is preferably receivable within the catheter.

Another aspect of the invention provides a catheter system including

a catheter;

a needle; and

a stylet;

the stylet including an elongate portion;

the elongate portion being receivable within the catheter;

the elongate portion and the catheter being co-operably configured such that, when the elongate portion is so received, at least a portion of the elongate portion is spaced from the catheter such that the at least portion and the catheter together define at least one elongate void for conveying fluid along the catheter at least one of to or from one or more sites within a patient;

the needle being receivable within the catheter to insert the catheter into the patient.

The stylet preferably includes a connector for sealingly engaging an end of the catheter external the patient to fluidly connect the void(s) with at least one of a fluid source or a fluid destination.

Optionally the catheter has one or more side openings positionable within the patient to fluidly connect the void(s) to one or more of the sites within the patient.

The system may be a nerve block catheter system or a blood vessel catheter system.

Preferably the system, of any of the foregoing aspects of the invention, is individually packaged in a package such that the system is sterile upon removal of the system from the package.

Another aspect of the invention provides a stylet for a catheter system;

the stylet including an elongate portion and a shaped portion;

the elongate portion being receivable within a catheter and configured such that, when the elongate portion is so received, at least a portion of the elongate portion is spaced from the catheter such that the at least portion and the catheter together define at least one elongate void for conveying fluid along the catheter at least one of to or from one or more sites within a patient;

the shaped portion being positioned to be within the patient.

Preferably the shaped portion has a shape complementary to a cylindrical interior of the catheter to obstruct the catheter.

Another aspect of the invention provides a method, of fluidly communicating with one or more sites within a patient, including

inserting into the patient a catheter;

inserting into the catheter a stylet.

The method preferably includes manipulating the stylet to position the shaped portion between openings, of the catheter, within the patient. The stylet may be manipulated to move the shaped portion so as to remove build-up, from the catheter, within the patient, e.g. to remove build-up, from an open end of the catheter, within the patient. Preferably the stylet is manipulated to move the shaped portion beyond an or the open end, of the catheter, within the patient. The method may include at least one of advancing and retracting the stylet to establish fluid communication via selected ones of a plurality of side openings along the catheter.

Another aspect of the invention provides a method, of fluidly communicating with one or more sites within a patient, including

inserting into the patient a catheter;

inserting into the catheter the stylet;

manipulating the stylet to remove build-up, from the catheter, within the patient.

The method preferably includes, after inserting the catheter, leaving for a period of time the catheter in place.

Another aspect of the invention provides a method, of fluidly communicating with one or more sites within a patient, including

inserting into the patient a catheter; and

inserting into the catheter a stylet configured such that, when so inserted, at least a portion of the stylet is spaced from the catheter such that the at least portion and the catheter together define the at least one elongate void for conveying fluid along the catheter at least one of to or from one or more sites within a patient; then

leaving for a period the stylet in place.

Preferably the period is at least one hour.

Optionally the inserting into the patient a catheter and the inserting into the catheter a stylet are during an attendance to the patient; and

the method further includes leaving the stylet and catheter in place until a subsequent attendance to the patient.

Another aspect of the invention provides a method, of fluidly communicating with one or more sites within a patient, including

inserting into the patient a catheter; then

obstructing flow through the catheter at a location within the patient.

Preferably the obstructing is at a location along the catheter to establish fluid communication via selected ones of a plurality of side openings along the catheter.

Preferably the inserting into the patient a catheter includes

inserting into the patient a needle externally carrying the catheter; and

withdrawing the needle.

Another aspect of the invention provides a method, of fluidly communicating with one or more sites within a patient, including

inserting into the patient a catheter;

inserting into the catheter a stylet;

wherein the inserting into the patient a catheter includes
inserting into the patient a needle externally carrying the catheter; and
withdrawing the needle.

Another aspect of the invention provides a method of administering a nerve block including

fluidly communicating with one or more nerves;

supplying at least one anesthetic to the nerve(s) via a catheter.

Another aspect of the invention provides a method of monitoring arterial blood pressure including fluidly communicating with a blood vessel, e.g. an artery.

Another aspect of the invention provides a method of removing build-up, from a catheter, within a patient,

the method including inserting into the catheter a stylet; and

manipulating the stylet.

Another aspect of the invention provides a stylet, for a catheter system, individually packaged in a package;

the stylet including an elongate portion;

the elongate portion being receivable within a catheter and configured such that, when the elongate portion is so received, at least a portion of the elongate portion is spaced from the catheter such that the at least portion and the catheter together define at least one elongate void for conveying fluid along the catheter at least one of to or from one or more sites within a patient;

the stylet and the package being sterile such that the stylet is sterile upon removal of the stylet from the package.

### BRIEF DESCRIPTION OF DRAWINGS

FIGS. 1 to 6 are schematic side views illustrating a preferred method of inserting and operating a preferred catheter system;

FIG. 7 schematically illustrates another catheter system in use;

FIG. 8 is a schematic cross section view of an arterial catheter system in situ;

FIG. 9 is a transverse cross section view of a catheter and a stylet;

FIG. 10 is a perspective view of a kinked catheter on test;

FIGS. 11 and 12 chart blood pressure signal attenuation as a function of kinking angle for various stylet sizes; and

FIGS. 13 to 17 are blood pressure wave forms comparing a catheter system on test to an unkinked stylet-less catheter.

### DESCRIPTION OF EMBODIMENTS

The catheter system of FIGS. 1 to 6 includes a catheter 10, a needle 20, and a stylet 30.

The catheter 10 includes an elongate thin walled tubular body 11 formed of suitably pliable polymer material. The distal (or forward) end of the body 11 terminates at simple open end 12. The other (or proximal or rearward) end of the body 11 terminates in a rearwardly flared, connector receiving, portion 14. In this example, the connector receiving portion is integrally formed with the body 11. Preferably, the portion 14 is a female luer connection.

To suit nerve blocking applications the catheter preferably has an outer diameter of 16 g to 20 g. Arterial line catheters are preferably 18 g to 22 g. In this example, the catheter has an outer diameter of 20 g (0.902 mm, 0.0355 in) and is dimensioned to accommodate a 22 g needle. To aid in insertion, the outer diameter may be conically tapered at the tip 12 to define a lead-in.

The needle 20 includes a needle body 21. The body 21 is a straight cylindrical tube of rigid metallic construction. In this example, the needle 20 is a short beveled nerve block needle. The distal end of the body 21 terminates at a penetrating tip 22, which in this example includes a single oblique planar face the edges of which are sharp.

The body 21 is receivable within the catheter 10 such that the catheter 10 is externally carried by the needle 20. The catheter 10 is fitted to the needle 20 in the manner of a sleeve. In particular, the portion 11 forms a close fitting sleeve about the body 21 and is supported by the body 21 during insertion into the patient.

The proximal end of the body 21 is rigidly mounted within a connector 23. The connector 23 includes a forwardly projecting cylindrical boss 24. The boss 24 is concentric with the body 21 and dimensioned to be snugly received within, and to sealingly engage, the connector receiving portion 14 of the catheter 10 to resist inadvertent separation of the needle 20 from the catheter 10.

A rearward end of the needle 20, or more specifically its connector 23, terminates in a port 25 co-operable with a tube to fluidly connect to the tube with an interior of the needle 21. In an alternate construction, a tube may be directly connected to the needle 21 during manufacture.

The stylet 30 includes an elongate portion in the form of a long cylindrical solid rod 31 formed of incompressible, non-porous, semi-rigid plastic or metal. For the avoidance of doubt, “rigid” and similar terms as used herein refer to material which does not deform appreciably in use, and “semi-rigid” is used in contrast to “freely pliable” to refer to materials which deform, but offer appreciable resistance thereto, in use.

The distal end of the rod 31 terminates in a shaped formation 32. The shaped formation 32 is integrally formed with the rod 31 but is distinct therefrom in that it has an appreciably different shape. The shaped formation 32 is a bulbous portion having a cylindrical exterior shaped to closely fit within the cylindrical interior of the catheter 10 (or more specifically its body 11). In this example, the formation 32 has a dome shaped rounded leading end.

The proximal end of the rod 31 is rigidly mounted within a connector 33. The connector 33 is preferably a female to male luer handle and includes a forwardly projecting conically tapered cylindrical boss 34 dimensioned to snugly fit within the connector receiving portion 14 to sealingly engage the catheter 10 and to resist inadvertent separation of the connector 33 and the catheter 10. The catheter 10 has a luer lock or similar mechanism to engage the stylet (i.e. the stylet screws into place with threads). The boss 34 is a male connector end.

The stylet, or more specifically its connector 33, rearwardly terminates in a flow port 35 co-operable with a tube to connect the stylet to a fluid source or fluid destination. In an alternate construction, a tube may be directly connected to the stylet during manufacture. The boss 34 is a tubular boss defining an outlet 36 at its forward end.

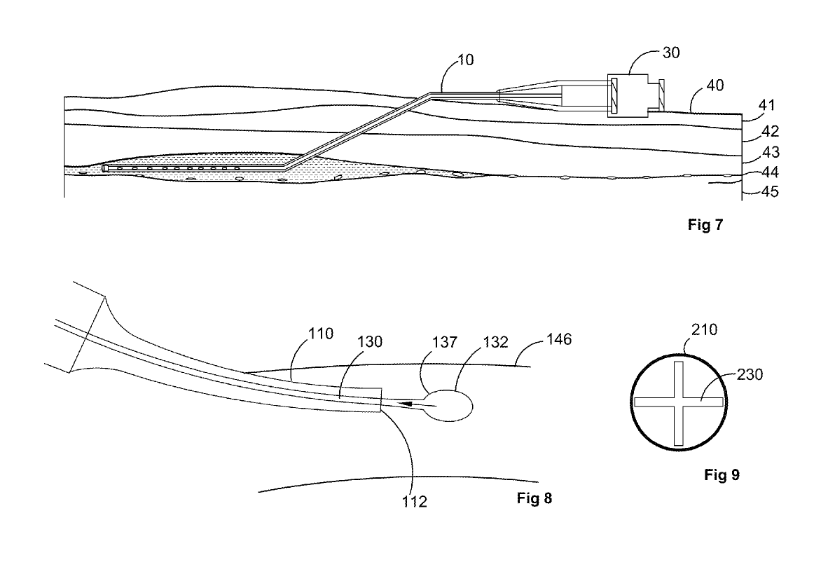

FIG. 7 schematically illustrates the catheter 10 and stylet 30 inserted into a patient 40 to perform a fascial plane nerve block called a transversus abdominis plane block. As illustrated the patient 40 includes (in order in an inwards direction) skin layer 41, external oblique layer 42, internal oblique layer 43, fascial plane 44 and transversus abdominis 45. The fascial plane 44 separates the internal oblique and the transversus abdominis and it is within this plane that the nerves lie.

To insert the catheter 10 and stylet 30, first the catheter 10 is inserted. The catheter 10 is fitted to the needle 20 as in FIG. 1 and manipulated to drive the piercing tip 22 and to manoeuvre the tip 22 and the distal end portions of the catheter 10 and needle 20 into the fascial plane. Positioning of the needle may be guided by electrical stimulation and/or ultrasound. By way of example, a voltage may be applied to the needle body 21 so that when its tip 22 (uninsulated by the catheter 10) acts on the nerves the patient observably twitches.

The configuration of the piercing tip 22 may be application dependent. Needles for arterial puncture are usually sharper (than needles for nerve blocks) having a Quinke tip or other tip more suited to vascular access.

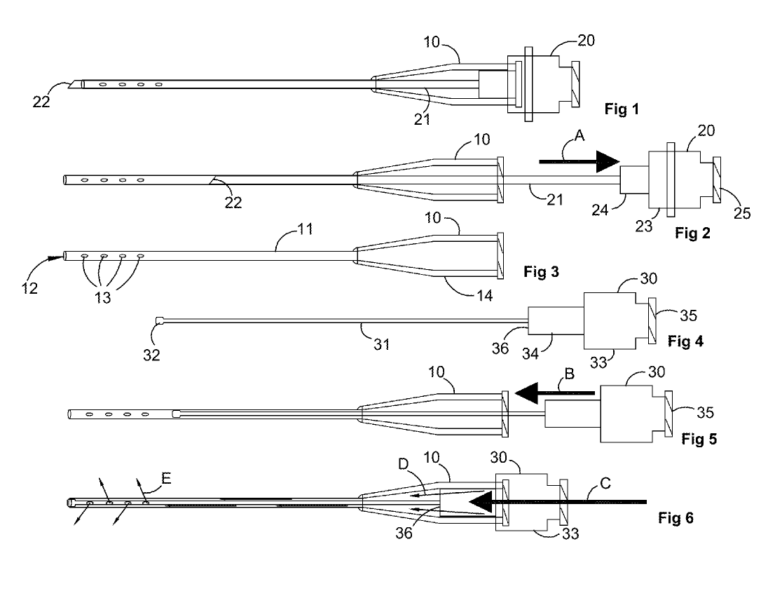

Once the subassembly 10, 20 is appropriately positioned, the needle 20 is withdrawn as suggested by arrow A in FIG. 2. The stylet 30 is then inserted into the catheter 10. The rounded leading end of the stylet 30 and the tapered interior of the portion 14 guides the stylet into the catheter 10, or more specifically its body 11.

In this example, the shaped formation 32 is dimensioned for a snug receipt within the body 11 so as to substantially occlude the body 11, although obstruction less than substantial occlusion would still be useful.

The catheter 10 and the stylet 30 are co-operably configured whereby when the stylet 30 is fully advanced the shaped portion 32 is brought into register with the open end 12 and the boss 34 sealingly engages the connector receiving portion 14. The cylindrical exterior of the rod 31 is of lesser diameter than the nominally cylindrical interior of the body 11 whereby a nominally annular void is defined between the exterior of the rod 31 and the interior of the body 11. Of course, given that the body 11 is flexible, this nominally annular void would in fact vary in shape along its length, and of course the portions of the void defined within the flared connector receiving portion 14 are larger than the void portions defined within the body 11.

The connector 33 defines one or more flow paths communicating the port 35 with the outlet 36 to fluidly communicate the port 35 with the nominally annular void.

Once the subassembly 10, 30 is in situ as in FIG. 7 the flexible tube of a fluid source comprising the tube and a syringe containing anaesthetic is fitted to the port 35. By advancing the plunger of the syringe fluid is driven through the connector 33 as suggested by arrow C to emerge from the connector 33 via the outlets 36 into the nominally annular void as suggested by arrows D. The fluid is in turn conveyed along the voids to the side openings 13 to emerge therefrom at respective sites within the fascial plane 44 or next to a nerve as suggested by arrows E.

The shaped formation 32 obstructs the open free end 12 of the catheter 10 and so promotes fluid flow through the side openings 13. In this example, the shaped formation 32 substantially occludes the open free end at 12 and causes outward flow through the side openings 13.

The described catheter over needle mode of insertion has been found to lead to more secure embedding of the catheter 10 within the patient (i.e. to have higher resistance to inadvertent withdrawal of the catheter) than conventional catheter through needle techniques.

The use of a stylet as described serves to not only reinforce the catheter 10 against kinking but also gives the anaesthetist a degree of control over the delivery pattern of anaesthetic within the patient. By way of example, the catheter 10 once inserted may be used without a stylet, i.e. a fluid source may be directly connected to the connector receiving portion 14. By operating the catheter 10 without the stylet 30 a high proportion of the supplied anaesthetic would be delivered to the patient via the open free end 12 of the catheter thus appreciably varying the delivery pattern. In other variants of the disclosed system and method, the delivery pattern may be varied by varying the location of the shaped portion 32 relative to the openings 12, 13. For example, in the illustrated variant, when the shaped portion 32 is inserted as illustrated, the side openings 13 are selected for fluid delivery and the open end 12 is deselected for fluid delivery. By more proximally positioning the shaped portion 32 distal ones of the openings 13 can be deselected. By way of example, if the shaped portion 32 were positioned half way along the group of four openings 13 only the proximal two of the openings 13 would be selected to convey fluid.

To so vary the position of the shaped portion 32 a set of stylets of varying length may be provided. Preferably each stylet of the set is individually packaged so that a selected one of the stylets can be used whilst the others remain sterile. Alternatively, the rod 31 may be mounted to slide through the connector 33 so that the spacing of the portion 32 from the connector 33 may be varied.

Alternatively the portion 32 may be advanced beyond the open end 12 to open the end 12 to flow.

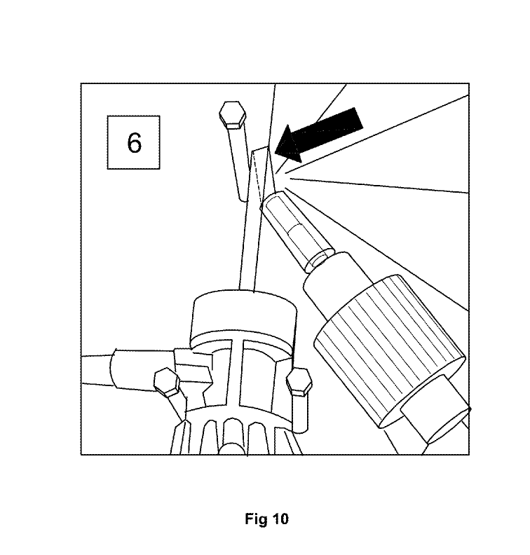

FIG. 8 schematically illustrates a catheter 110 and stylet 130, both parts of an arterial catheter system, in situ in an artery 146. The stylet 130 includes a connector (not shown) akin to the connector 33 to seal the arterial catheter 110 and allow fluid to flow up and down the arterial catheter. By way of example, the catheter may be used to deliver medicament, draw a blood sample, or simply for beat-to-beat blood pressure measurement.

As the skilled person will understand from the foregoing, to so monitor blood pressure, the catheter is connected to a suitable pressure sensor external to the patient.

Early testing suggests that the use of the stylet whilst monitoring blood pressure is a radical improvement over existing stylet-less approaches. In particular, this testing suggests:
the problems associated with the catheter kinking are substantially avoided;
the fidelity of the system including a 20 g catheter can be maintained by using a stylet no bigger than about 0.3 mm in diameter along its elongate, void-defining, portion;
the system with the stylet in place allows for a flow rate adequate for blood sampling; and
that even stylets with occluding ends can be passed into the catheter even when there is considerable pressure and flow out the catheter as the stylet is placed.

When blood pressure is monitored in this way, fluid may be delivered to the artery 46 via the catheter 110 to resist clotting within the catheter. Preferably the fluid is saline and is most preferably delivered very slowly, e.g. at about 3 mL/hour. Optionally the flushing fluid may include an anti-coagulant such as Heparin, although generally this is not the preferred approach.

Despite such precautions, build-up in the form of clotting can occur about the catheter tip 112. To address this build-up, the stylet 130 includes a distinct shaped portion 132 and is configured for that shaped portion to extend beyond the open end 112. The stylet 130 is longer than the catheter 110, long enough (relative to the catheter 110) to so clean the open end of the catheter whilst a portion of the stylet remains external the patient to be manipulated by hand. By periodically withdrawing the stylet 130, the shaped portion 132 is moved to act upon any such build-up causing it to break up and safely move away along the artery before it occludes the catheter 110 or grows to a dangerous size. Optionally, the stylet 130 may be fully withdrawn and replaced by another stylet. So replacing the stylet refreshes the interior of the catheter lumen.

The stylet might be so manipulated and replaced periodically, e.g. daily. For this purpose, separately packaged stylets and kits of multiple (potentially identical) stylets are contemplated. By way of example, a catheter system may include a kit made up of three individually packaged stylets to be used over the three days following the initial insertion of the catheter.

To emphasise, whilst the exemplary stylet 130 is longer than the catheter 110 and includes a bulbous end portion 132 to clean the catheter's open end, other useful variants are possible. By way of example:
shorter stylets may be used to clean the interior of the catheter lumen;
a stylet without a bulbous end portion may be manipulated to remove build-up; and
similar modes of cleaning may be applied to catheters having closed ends.

About 0.3 mm diameter is thought to be a practical minimum diameter for the elongate, void-defining, portion of stylets formed of plastics. Smaller diameters are thought to be practical in metallic stylets.

Smaller diameters present less restriction to flow, which for example can lead to improved blood pressure measurement. On the other hand, smaller diameters are more fragile. Hence the optimum diameter will depend on the application and in particular on the strength and flow rate requirements of the application. Generally speaking:
longer stylets must be thicker to be sufficiently robust; and
the fluid path for nerve block applications can be a lot smaller than for arterial blood pressure measurement.

For longer nerve block applications (such as the transversus abdominis block), the stylet could be up to 1 mm or so in external diameter to fit down a 16 g to 18 g catheter.

The rearward portion 137 of the shaped formation 132 rearwardly converges to define a lead-in surface to guide the shaped formation 132 back into the catheter 110 after it has been extended beyond the open end of the catheter 110. In this example, the bulbous end formation 132 has a continuous smoothly curved exterior to minimise build-up on the formation 132.

The described catheter systems may be installed and removed from the patient during a single attendance to the patient, although it is preferred that the catheter remain in place for ongoing use during a lengthy surgery, or even during the patient's entire hospital stay. By way of example, the described arterial catheter 110 and its stylet 130 may be left in place to provide continuous monitoring of arterial blood pressure over a period of days, or if ongoing monitoring is not required the connection arrangement (not shown) external the patient may simply be capped.

The installed components 110, 130 of the disclosed catheter system may advantageously be left in place well beyond their initial installation. By way of example, the catheter may be left in place and then removed during a subsequent attendance to the patient prior to leaving hospital, or even removed by the patient after leaving the hospital.

The stylet may be produced separately, e.g. to suit existing catheters.

### EXPERIMENTAL VALIDATION

The present inventor has tested various catheter systems to address the following questions:

1. How may the stylet affect the ease of injection/aspiration through the combined stylet/catheter system (for bolus and infusion in nerve block applications and blood sampling in arterial line applications)?

2. How does the stylet damp the arterial pressure waveform, and can it reduce the damping effect of kinking on the measured waveform? What is the optimal size of stylet to minimise damping and maintain kink resistance?

3. Is it feasible to place the arterial stylet down the catheter when arterial blood pressure is resisting passage of the stylet?

### Method and Apparatus

In view of the movement of blood during blood pressure measurement noted above, the present inventor recognised that it was important to test the catheter systems with a fluid that has a similar viscosity to blood, so that the resistance of the fluid moving in and out the end of the system is similar to how it would behave in the artery and bloodstream of a patient.

The viscosity of blood is typically between three to four times that of water, depending on the concentration of blood factors, particularly the concentration of red cells. To simulate blood, a solution of 30% sucrose, dyed with methylene blue to make it easily identifiable, was prepared. According to standard tables, this solution has a viscosity of 3.19 times that of water at 20 degrees Celsius.

An apparatus was constructed to produce the required pressure for testing of flow and to create a pressure waveform to simulate the arterial pulse. The “arterial” pulse was then measured simultaneously through the test and control catheters. The pressure generating system included a variable speed electric motor connected to a cam. Rotation of the cam was translated through a flexible lever into pressure on and movement of the barrel of a syringe. The syringe was filled with the sucrose solution which was transmitted via a tube to the testing chamber in which the ends of the test catheters were placed via sealing rubber membranes. The test catheters were further stabilised in position with epoxy resin to prevent inadvertent withdrawal. The catheters were positioned within the sucrose solution which was flushed between each experimental run to ensure it was not contaminated by the 0.9% NaCl (normal saline) flush solution from the pressure measuring system. Connected to the pressure generating syringe was a vertically mounted adjustable syringe on a 3-way connector, which, by allowing the sucrose solution to enter and compress the air in the syringe, acted to increase and decrease the pulse pressure generated by changing the compliance of the system.

The pressure measuring system consisted of two commercially available pressure measuring transducers (Edwards Laboratories), the whole system as essentially used for clinical measurement of blood pressures including the flush of low volumes of normal saline through a slow flush valve. The transducers were mounted on a board with 3-way connectors and tubing between the transducers to enable the simple switching of the pressure tubing from connection with one transducer to the other without the physical disconnection and reconnection of tubing. This crossover allowed the pressure from each catheter to be alternately measured by the alternative transducer. The signal from the transducers was displayed on a standard clinical monitor using simultaneous waveforms in different colours. The numerical readout gave the values of peak (systolic), trough (diastolic) and mean pressure after a short averaging interval. The pressure waveforms and measurements were recorded by digital photography for analysis. After flushing the system to remove air, the transducer flush valve was sealed off by closing a 3-way tap to prevent any fluid loss through back pressure on the valve or flush through and contamination of the sucrose solution with normal saline.

The ideal pressure transduction system has characteristics of low compliance to minimise movement of blood and normal saline back and forth within the system, as well as a high natural resonant frequency to enable accurate measurement of rapidly changing pressures. These physical characteristics of the transducer and catheter systems mean they are most accurate measuring small pressure changes at lower frequencies of change.

The major concern of adding the stylet into the system is that it may impair the movement of fluid within the measuring system, causing damping of the trace. It was therefore decided to test the operation of a variety of sizes of stylets at high frequencies and with relatively large pressure changes to accentuate the effect of any reduction in fidelity. The motor was therefore adjusted to produce a pressure wave (pulse) frequency of 120 to 150 (normal 80-100/min) per minute and the compliance adjusted to a pulse pressure of 90-120 mm Hg (normal 40-80).

Prototype stylets were prepared using commercially available nylon line of diameter between 0.17 and 0.6 mm (Maxima GmbH, Geretsried Germany) mounted into a luer lock fitting male/female combi-stopper (Braun) with a drilled fluid bypass hole of 1.5 mm diameter, that diameter selected to be a non-limiting part of the pressure and fluid channel compared to the 0.7 mm diameter of the 20 g catheter. An occluding bulb was created when needed using a small amount of resin on the end of the prototype stylet.

### Investigations Included

1. Fluid flow test, measurement of passive flow through the catheter stylet system to simulate blood sampling via the catheter and nerve block injections. A 10 ml syringe was placed vertically connected to a 3-way tap at the hub of the test catheter. The pressure was adjusted to 80 mm Hg to approximate normal mean arterial pressure and the time to fill the syringe to the 5 ml mark was recorded.

2. Damping and Kink Resistance Test. Assessment of the optimal size of stylet was done by measuring the damping effect of stylets of various sizes on the measurement of simulated arterial pressure. 20 g Optiva™ and 20 g Arrow™ catheters were tested with a variety of nylon stylets from 0.17 mm to 0.6 mm diameter. The test catheter and stylet was progressively kinked from straight (0 degrees) through a right angle (90 degrees) to kinked as much as possible (180 degrees). FIG. 10 illustrates an Optiva™ catheter with a stylet kinked to 180 degrees (at a point suggested by the black arrow).

FIG. 10 also shows the test apparatus including a rudimentary protractor with a nail adjacent its point of convergence. The nail serves as a stop to assist in manipulating the catheter system.

Simultaneous pressure readings from the test and control catheters (i.e. catheters without any stylet in place) were obtained using separate transducers and displayed on a monitor screen. The transducers were swapped between the control and the stylet containing catheters and then the result averaged. An average percent reduction in pulse pressure was calculated (systolic-diastolic pressure of test catheter divided by systolic-diastolic pressure of control catheter*100) and recorded for each diameter of stylet.

3. Insertion Test. For ease of use, the stylet needs to be passed relatively easily into the catheter when it is in the artery, against the pressure of blood in the vessel. This was assessed using the previously determined optimal sized stylet. A prototype nylon stylet was made with a near occluding bulb on the end to just fit through a 20 g catheter. The pressure in the catheter was adjusted to 200 mm Hg by moving the height of the reservoir of 30% sucrose solution. While the sucrose solution was free flowing from the catheter, the nylon stylet with a bulb on the tip was passed into the catheter and any difficulty in insertion noted.

### Results

1. Fluid Flow Test

A. 20 g Optiva ™ catheter

| Stylet | Time to fill to 5 ml |
|---|---|
| Control no stylet | 13.5 sec |
| 0.6 mm | 68 secs for 1 ml |
| 0.42 mm | 45 secs |
| 0.37 mm | 36 secs |
| 0.30 mm | 24 secs |
| 0.25 mm | 26 secs |
| 0.17 mm | 19 secs |

B. 20 g Arrow ™ Catheter

| Stylet | Time to fill to 5 ml |
|---|---|
| Control no stylet | 15 sec |
| 0.6 mm | 107 secs for 1 ml |
| 0.42 mm | 50 secs |
| 0.37 mm | 37 secs |
| 0.30 mm | 32 secs |
| 0.25 mm | 28 secs |
| 0.17 mm | 24 secs |

2. Damping and Kink Resistance Test

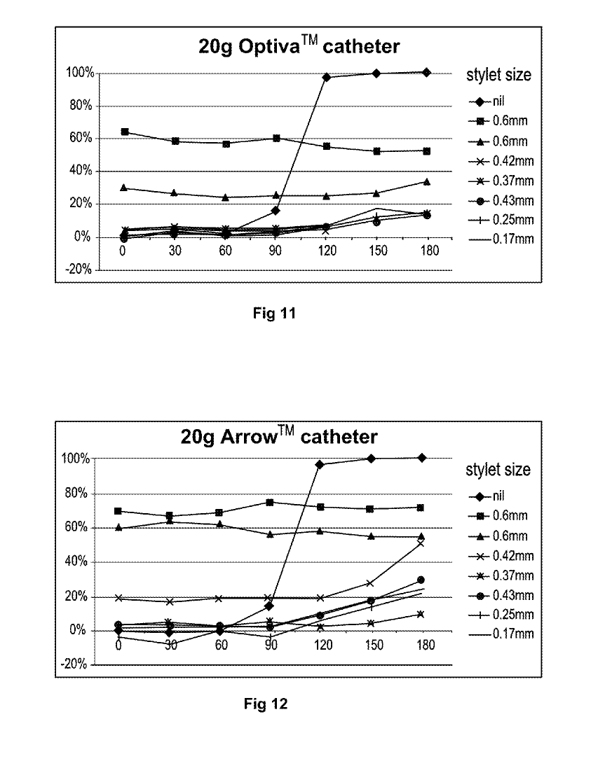

FIGS. 11 and 12 chart the reduction in the measured pulse pressure (as a percentage of the pulse pressure measured using the control catheter) plotted on the vertical axis as a function of the angle to which the catheter is kinked on the horizontal axis (from 0 degrees to, fully kinked, 180 degrees). The legends indicate stylet diameters from nil (no stylet) to 0.6 mm

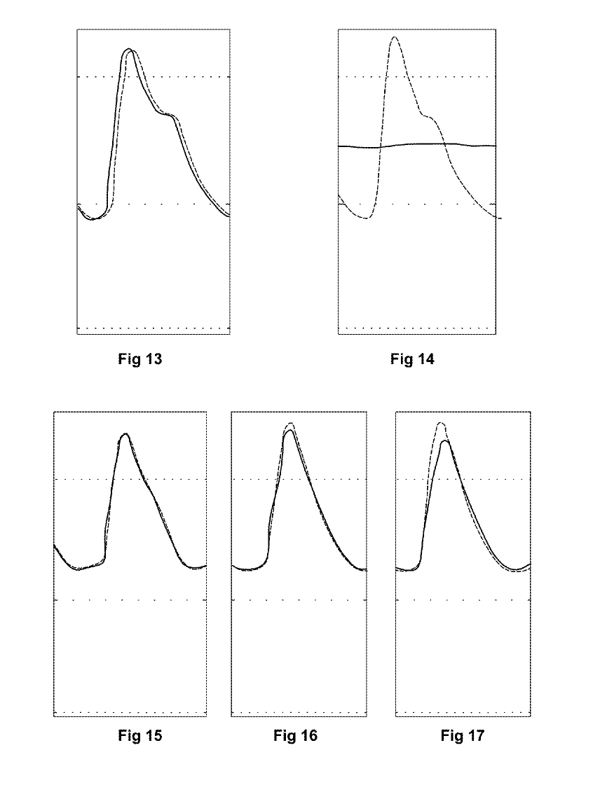

FIGS. 13 to 17 show measured pressure waves obtained using 20 g Optiva™ catheters. Each of these Figures compares the pressure wave (shown in solid line) obtained from a test catheter to the pressure wave (shown in dotted line) obtained from an unkinked stylet-less control catheter.

In FIG. 13 the catheters are straight (i.e. the angle of kinking is 0 degrees). The test catheter is not fitted with a stylet and is thus nominally identical to the control catheter. Thus, FIG. 13 gives an indication of the expected degree of experimental error. FIG. 14 shows the result of the same stylet-less test catheter being kinked to 120 degrees: the pressure wave is almost entirely attenuated.

In contrast FIGS. 15, 16 and 17 respectively show the results of kinking to 0 degrees, 120 degrees and 180 degrees when a Ø0.3 mm stylet is in place. FIG. 15 shows that when the catheter is straight (as ideally it should be in use), the stylet causes no discernible deterioration of the signal.

A comparison of the FIGS. 14 and 16 demonstrates the benefits of the stylet. With the stylet in place, a trace of the pressure wave can be obtained that matches the trace obtained from a straight catheter despite a kinking condition (i.e. 120 degrees) at which the signal from a stylet-less catheter would be almost entirely lost. The obtained trace matches the “straight catheter trace” within the degree of experimental error contemplated in FIG. 13.

FIG. 17 illustrates that even when the catheter is fully reversed on itself (i.e. kinked to 180 degrees), a meaningful trace of the pressure wave can be obtained.

In the 20 g Optiva™ catheter, all the stylets of 0.42 mm diameter and less performed almost identically. In the 20 g Arrow™ catheter, the 0.42 mm stylet was also damped relative to the smaller stylets. The free flow test showed that all the stylets slowed flow; in both catheters the 0.37 mm stylet slowed the flow by approximately a third and the 0.3 mm stylet slowed the flow by approximately half. Both these outcomes would be clinically acceptable, although the less reduction in flow the better.

For both the pressure test and the flow test, a smaller stylet is an advantage, however in use the kink resistance and placement is likely to be better with the widest diameter stylet. The 0.37 mm and 0.3 mm stylets were the largest stylets that did not show significant damping on the blood pressure transduction test and both were similar in the test, kinking up to 180 degrees. Of these, the 0.3 mm stylet is preferred because it is smaller and will be less likely to obstruct the catheter in clinical conditions.

3. Insertion Test

The 0.3 mm stylet was modified with a small bulb of glue on the end to create a near occluding end then tested in the 20 g Optiva™ catheter. The sucrose solution was placed at a height of 220 cm which creates a driving pressure of approximately 175 mm Hg. The sucrose solution was allowed to flow freely from the hub of a 20 g Optiva™ catheter inserted into the pressurised solution. The 0.3 mm stylet with bulb was inserted on the first attempt into the 20 g catheter. Once the bulb was in the catheter lumen and largely occluding it, the insertion proceeded smoothly.

### Discussion

This testing demonstrates the marked effect the intra-catheter stylet has on pressure measurement of a simulated arterial pulse via commonly used arterial catheters. The area of lumen required for an accurate measurement of blood pressure is likely to be influenced by the shape of the lumen available. A stylet of 0.6 mm diameter in a 20 g lumen of 0.7 mm occupies 73% of the available area while a stylet of 0.55 mm occupies 62%, 0.42 mm occupies 36%, 0.37 mm occupies 28%, 0.3 mm occupies 18%, 0.25 mm occupies 13% and the 0.17 mm stylet occupies only 6% of the available area.

The fluid flow test showed that all the stylets of 0.42 mm and below showed an acceptable rate of passive flow in these 20 g catheters, with greater flow for the smaller stylets. Even the smallest stylet of 0.17 mm diameter occupying only 6% of the lumen reduced flow by approximately a third with the 0.42 mm stylets reducing flow by 70%. These figures would all be acceptable clinically as blood sampling time usually only takes a few minutes, mostly in setting up the sampling, labelling and other tasks, while withdrawing the blood is a relatively small portion of the task.

All stylets reduced the damping effect of kinking the catheter, although the larger stylets over 0.42 mm diameter also produced significant damping by their presence. All the stylets of 0.37 mm and under performed very similarly in the damping and kinking test. The 0.42 mm stylet showed a reduction in performance with kinking in the Arrow™ catheter, particularly at over 90 degrees of kinking. This may be because the Arrow™ catheter is softer than the Optiva™ and kinks differently, the introducer needle is slightly smaller than the Optiva™ suggesting the lumen may be smaller, and it is slightly longer which may accentuate any damping from the stylet. There was no lower limit seen to the effect of the stylet maintaining a lumen for pressure transduction during kinking over the stylets measured. The stylet may maintain a lumen by resisting kinking and therefore maintaining a lumen around the unkinked stylet. This effect was seen for the biggest stylets, however they occluded so much of the lumen that the trace was damped in all positions. The smaller stylets allowed the catheter to apparently kink in a similar fashion to the stylet-less catheter with the opposite walls coming together to occlude the fluid path. The position of the stylet between the walls however is thought to have made a smaller lumen each side of the stylet where the walls of the catheter were unable to oppose. Using the simulated blood in this testing, this reduced lumen remained adequate for blood pressure measurement with only a small reduction in measured pulse pressure even with extreme kinking which would be very valuable clinically where the arterial catheter has been kinked for example by movement of the patient.

The practical lower limit for the size of a stylet would be determined by the stiffness of the stylet required to insert it into an arterial catheter placed into an artery. A plain stylet would be inserted much more easily than one with a bulb on the end, however the bulb confers other benefits, particularly the ability to clear debris and clot out of the lumen of the catheter on removal. The 0.17 mm stylet was very fine and would be impractical to insert against blood coming from and arterial cannula in situ unless it was very stiff, probably a steel wire. A plastic stylet would need to be larger than a steel stylet as it is less rigid, however clinicians would be more likely to accept a plastic stylet as it would probably conform better to the patient's anatomy and be less likely to perforate and damage arteries, nerves or other structures.

The 0.3 mm stylet was chosen for the insertion test as a compromise, as the area of the stylet is only half that of the 0.42 mm stylet with better performance on the fluid flow, damping and kinking test. If the plastic was suitably rigid, the stylet size could be reduced further. The insertion test showed that the 0.3 mm stylet could be feasibly inserted into a catheter with free flowing arterial blood under pressure coming out. In practice, most clinicians occlude the artery and catheter tip proximally to prevent the free flow of blood from the catheter during connection of the transducer line. This would aid insertion of the stylet as the only time the stylet would be subject to being pushed against the full pressure of arterial blood is when the bulb reaches the very end of the catheter in which position the stylet is maximally supported by the catheter walls, enabling greater force to be transmitted to the bulb on the tip.

1-46. (canceled)

47. A stylet for a catheter system, the stylet including:
an elongate portion and a connector;
the elongate portion being receivable within a catheter and configured such that, when the elongate portion is so received, at least a portion of the elongate portion is spaced from the catheter such that the at least portion and the catheter together define at least one elongate void;
wherein the elongate void(s) is configured to run from external the patient to within the patient to convey fluid along the catheter at least one of to or from one or more sites within a patient; and
the connector is configured to sealingly engage the catheter external the patient and define one or more flow paths open to the void(s).

48. The stylet of claim 47 including a shaped portion positioned to be within the patient and shaped to obstruct flow through the catheter.

49. The stylet of claim 48, wherein the shaped portion has a shape complementary to a cylindrical interior of the catheter.

50. The stylet of claim 48, wherein the shaped portion is in substance at an end of the stylet.

51. The stylet of claim 48, wherein at least a rearward portion of an exterior of the shaped portion rearwardly converges.

52. The stylet of claim 47, wherein the catheter is a flexible catheter capable of kinking when the elongate portion is so received; and
the stylet is configured to prevent occlusion of the catheter due to the kinking.

53. The stylet of claim 47 individually packaged in a package such that the stylet is sterile upon removal of the stylet from the package.

54. A catheter system including:
a catheter; and
a stylet including an elongate portion and a connector; the elongate portion being receivable within a catheter and configured such that, when the elongate portion is so received, at least a portion of the elongate portion is spaced from the catheter such that the at least portion and the catheter together define at least one elongate void;
wherein the elongate void(s) is configured to run from external the patient to within the patient to convey fluid along the catheter at least one of to or from one or more sites within a patient; and
the connector is configured to sealingly engage the catheter external the patient and define one or more flow paths open to the void(s).

55. The system of claim 54 further including a needle receivable within the catheter to insert the catheter into the patient.

56. The system of claim 55, wherein the catheter has an open end and the stylet is longer than the catheter to project beyond the open end whilst the open end remains open to flow.

57. The system of claim 54, wherein the catheter is a flexible catheter.

58. The system of claim 54 being a nerve block catheter system.

59. The system of claim 54 being a blood vessel catheter system.

60. The system of claim 54 individually packaged in a package such that the system is sterile upon removal of the system from the package.

61. A catheter system including:
a catheter;
a needle;
a stylet; and
a connector;
the stylet including an elongate portion; the elongate portion being receivable within the catheter; the elongate portion and the catheter being co-operably configured such that, when the elongate portion is so received, at least a portion of the elongate portion is spaced from the catheter such that the at least portion and the catheter together define at least one elongate void for conveying fluid along the catheter at least one of to or from one or more sites within a patient;
the needle being receivable within the catheter to insert the catheter into the patient;
the connector being configured to be external to the patient and to define one or more flow paths open to the elongate void(s).

62. A catheter system including:
a catheter; and
a stylet; the stylet including an elongate portion; the elongate portion being receivable within the catheter; the elongate portion and the catheter being co-operably configured such that, when the elongate portion is so received, at least a portion of the elongate portion is spaced from the catheter such that the at least portion and the catheter together define at least one elongate void for conveying fluid along the catheter at least one of to or from one or more sites within a patient;
the catheter being a flexible catheter capable of kinking when the stylet is so inserted; and the stylet being configured to prevent occlusion of the catheter due to the kinking.

63. The system of claim 62 including a connector configured to be external to the patient and to define one or more flow paths open to the at least one elongate void.

64. A catheter system for fluidly communicating, via at least one elongate void, with one or more sites within a patient, the system including:
a catheter having an open end positionable within the patient;
a stylet; and
a connector;
the stylet including an elongate portion and a shaped portion;
the elongate portion being receivable within the catheter; the elongate portion and the catheter being co-operably configured such that, when the elongate portion is so received, at least a portion of the elongate portion is spaced from the catheter such that the at least portion and the catheter together define the at least one elongate void;
wherein the elongate void is configured to run from external the patient to within the patient to convey fluid along the catheter at least one of to or from the one or more sites within a patient;
the shaped portion is positionable within the patient beyond the open end and shaped to act upon build-up about the open end such that the stylet is withdrawable to remove the build-up; and
at least a rearward portion of an exterior of the shaped portion rearwardly converges to guide the shaped portion into the catheter; the connector being configured to be external to the patient and to define one or more flow paths open to the elongate void(s).

65. The system of claim 64, wherein the shaped portion is a bulbous portion.

66. The system of claim 64, wherein the shaped portion is in substance at an end of the stylet.
